(19) United States
(12) Patent Application Publication
(10) Pub. No.: US 2012/0093887 A1
(43) Pub. Date: Apr. 19, 2012

(54) AMORPHOUS VARENICLINE TARTRATE CO-PRECIPITATES

(75) Inventors: Krishnadatt Sharma, Maharashtra (IN); Arvapally Seshu Kumar, Andhra Pradesh (IN); Nitin Sharadchandra Pradhan, Maharashtra (IN)

(73) Assignee: ACTAVIS GROUP PTC EHF, Hafnarfjordur (IS)

(21) Appl. No.: 13/322,277

(22) PCT Filed: Jun. 9, 2010
(86) PCT No.: PCT/IB10/01608

§ 371 Date: Jan. 6, 2012

(30) Foreign Application Priority Data
Jun. 10, 2009 (IN) 1364/CHE/2009

(51) Int. Cl. A61K 9/14; A61P 25/00; A61P 1/00; A61P 21/00; A61P 25/22; A61P 9/00; A61P 25/24; A61P 25/18; A61P 25/34; A61K 31/4985; A61P 29/00

(52) U.S. Cl. 424/400; 514/250

(57) ABSTRACT

Disclosed herein is a stable amorphous coprecipitate comprising varenicline tartrate and a pharmaceutically acceptable excipient selected from the group consisting of maltodextrin, lactose monohydrate and 2-hydroxypropyl-β-cyclodextrin, method for the preparation, pharmaceutical compositions, and method of treating thereof. Advantageously, the amorphous coprecipitates of varenicline tartrate disclosed herein have improved physiochemical characteristics that assist in the effective bioavailability.

### CROSS REFERENCE TO RELATED APPLICATION

This application claims the benefit of priority to Indian provisional application No. 1364/CHE/2009, filed on Jun. 10, 2009, which is incorporated herein by reference in its entirety.

### FIELD OF THE DISCLOSURE

Disclosed herein are stable amorphous co-precipitates of varenicline tartrate with pharmaceutically acceptable excipients, methods for the preparation, pharmaceutical compositions, and methods of treating thereof.

### BACKGROUND

Varenicline, 5,8,14-triazatetracyclo [10.3.1.0^{2,11},0^{4,9}]hexadeca-2(11),3,5,7,9-pentaene, is known to bind to neuronal nicotinic acetylcholine specific receptor sites and is useful in modulating cholinergic function. This compound is useful in the treatment of inflammatory bowel disease, irritable bowel syndrome, spastic dystonia, chronic pain, acute pain, vasoconstriction, anxiety, panic disorder, depression, cognitive dysfunction, drug/toxin-induced cognitive impairment (e.g., from alcohol, barbiturates, vitamin deficiencies, recreational drugs, lead, arsenic, mercury), particularly, nicotine dependency, addiction and withdrawal; including use in smoking cessation therapy. Varenicline is represented by the following structural formula:

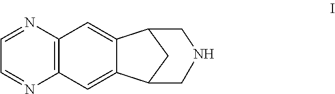

and its first synthesis was disclosed in U.S. Pat. No. 6,410,550 (hereinafter referred to as the '550 patent). Varenicline is sold by Pfizer under the brand name CHANTIX™ to help adults quit smoking by blocking α₄β₂ nicotinic acetylcholine receptor subtypes. It is orally administered as tablets containing 0.85 mg or 1.71 mg of varenicline tartrate equivalent to 0.5 mg or 1 mg of varenicline.

The '550 patent describes various processes for the preparation of aryl fused azapolycyclic compounds, including varenicline, and their pharmaceutically acceptable salts, combinations with other therapeutic agents, and methods of using such combinations in the treatment of neurogical and psychological disorders. Varenicline has been exemplified as a free base and a hydrochloride salt in the '550 patent.

U.S. Pat. No. 6,890,927 (hereinafter referred to as the '927 patent) discloses tartrate salts, including L-tartrate, D-tartrate, D,L-tartrate and meso-tartrate, of varenicline and their polymorphs, processes for their preparation, and pharmaceutical compositions thereof. The '927 patent further discloses various polymorphs of the varenicline L-tartrate salt, including two anhydrous polymorphs (Forms A & B) and a hydrate polymorph (Form C), and characterizes them by powder X-ray diffraction (P-XRD), X-ray crystal structure, solid state ¹³C NMR spectroscopy, and Differential Scanning Calorimetry (DSC).

Varenicline tartrate, 7,8,9,10-tetrahydro-6,10-methano-6H-pyrazino [2,3-h][3]benzazepine, (2R,3R)-2,3-dihydroxybutanedioate (1:1), has a molecular weight of 361.35 Daltons, and a molecular formula of C₁₃H₁₃N₃.C₄H₆O₆. Varenicline tartrate is represented by the following structural formula:

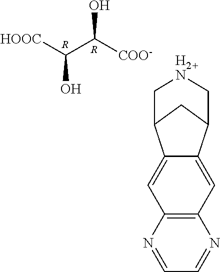

U.S. Pat. No. 6,787,549 discloses citrate salt of varenicline and its polymorphic forms including hydrate (Form A), anhydrous or nearly anhydrous form (Form B), processes for their preparation, and pharmaceutical compositions thereof.

U.S. Pat. No. 6,794,388 discloses the succinate salt of varenicline and its polymorphic forms including hydrate and anhydrous forms, processes for their preparation, and pharmaceutical compositions thereof.

PCT Publication No. WO 2008/060487 (hereinafter referred to as the '487 application) discloses crystal forms of intermediates used in the process for the preparation of varenicline tartrate, including the varenicline free base. According to the '487 application, the varenicline free base exists in four crystalline forms (Form A, Form C, Form D and Form E). The '487 application further describes a process for preparing substantially pure varenicline free base crystalline form C suitable for administration to a human subject comprising a) less than 2% by weight of N-formylvarenicline, and b) less than 2% by weight of N-carboxyvarenicline adduct, comprising the step of crystallizing varenicline from the crystallization solvent or solvent combination comprising an organic non-chlorinated solvent, wherein the crystallization solvent or solvent combinations used to isolate substantially pure varenicline free base form C is an organic non-chlorinated solvent selected from the group consisting of toluene, xylene, hexane, cyclohexane, heptane, octane, nonane and decane.

PCT Publication No. WO 2009/109651 (hereinafter referred to as the '651 application) discloses various crystalline salt forms of varenicline, including varenicline hemi-adipate (Form I), fumarate (Form I), glutarate (Form I), glycolate (Form I), hydrochloride (Forms I, and III), α-ketoglutarate (Form I), L-malate (Forms I, II, III, and IV), maleate (Form I), malonate (Form I), DL-mandelate (Form I), di-(methane sulfonate) (Form I), oxalate (Form I), phosphate (Forms I, II, and III), S-2-pyrrolidinon-5-carboxylate (Form I), galactarate (Form I), DL-lactate (Form I), hemi-1,2-ethane disulfonate (Form I), and hemi-L-lactate (Form I); and characterizes them by powder X-ray diffraction (P-XRD) and IR spectroscopy; processes for their preparation; and pharmaceutical compositions thereof.

PCT Publication No. WO 2009/111623 (hereinafter referred to as the '623 application) discloses the amorphous form of varenicline tartrate, amorphous solid dispersions of varenicline tartrate and a pharmaceutical carrier, and processes for the preparation thereof. While the '623 application mentions that the varenicline tartrate can form amorphous solid dispersions with various pharmaceutically acceptable carriers, such as, for example, hydrophilic carriers like polymers of N-vinylpyrrolidone, commonly known as polyvinyl pyrrolidines, gums, cellulose derivatives, cyclodextrins, gelatins, hypromellose phthalate, sugars, polyhydhc alcohols, polyethylene glycol, polyethylene oxides, polyoxyalkylene derivatives, methacrylic acid copolymers, polyvinylalcohols, and propylene glycol derivatives; only the solid dispersions of varenicline tartrate with hydroxypropyl cellulose, hydroxypropyl methylcellulose and povidone had been prepared and/or isolated.

U.S. Patent Application No. 2009/0215787 (hereinafter referred to as the '787 application) discloses the amorphous form and three crystalline polymorphs (Form D, Form E & Form F) of varenicline tartrate, processes for the preparation, and characterizes them by powder X-ray diffraction (P-XRD), Fourier transform Infrared spectroscopy (FT-IR), differential scanning calorimetry (DSC) and thermogravimetric analysis (TGA). The '787 application further teaches that the varenicline tartrate can form amorphous solid dispersions with various pharmaceutically acceptable carriers, such as, for example, polyethylene glycols (PEG), polyvinylpyrrlidones (PVP), sugars, lactose, starches, mannitol, methylcellulose, hydroxylethylcellulose, hydroxylmethylcellulose, ethylcellulose, hydroxylpropylmethylcellulose (HPMC) or other cellulose derivatives, α-cyclodextrin, β-cyclodextrin and hydroxylpropyl-β-cyclodextrin; but only the solid dispersions of varenicline tartrate with polyvinylpyrrlidone had been prepared and/or isolated.

An important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered pharmaceutical compound may reach the patient's bloodstream. The rate of dissolution is a consideration in formulating syrups, elixirs and other liquid medicaments. The solid state form of a compound may also affect its behavior on compaction and its storage stability.

It has been disclosed in the art that the amorphous forms of a number of pharmaceutical compounds exhibit superior dissolution characteristics and in some cases different bioavailability patterns compared to crystalline forms [Konno T., Chem. Pharm. Bull., 38, 2003 (1990)]. For some therapeutic indications, one bioavailability pattern may be favored over another.

The discovery of new solid state forms of a pharmaceutical compound provides a new opportunity to improve the performance characteristics of a pharmaceutical product. It enlarges the repertoire of materials that a formulation scientist has available for designing, for example, a pharmaceutical dosage form of a pharmaceutical compound with a targeted release profile or other desired characteristic.

### SUMMARY

The present inventors have carried out extensive experimentation to prepare amorphous co-precipitates of varenicline tartrate with different pharmaceutically acceptable excipients, in different ratios, such as povidone K30 (1:10, 1:20 and 1:1), lactose monohydrate (1:10), mannitol (1:10), maltodextrin (1:10), D-maltitol (1:10), 2-hydroxypropyl-β-cyclodextrin (1:10), dextrate hydrate (1:10), xylitol (1:10) and sorbitol. It has been surprisingly and unexpectedly found that the amorphous co-precipitates of varenicline tartrate are formed only with povidone K30, lactose monohydrate, maltodextrin, 2-hydroxypropyl-β-cyclodextrin, whereas the varenicline tartrate does not form amorphous co-precipitates with mannitol, D-maltitol, dextrate hydrate, xylitol and sorbitol. The products obtained after removal of solvent from the solvent solution containing varenicline tartrate and the excipients such as mannitol, D-maltitol, dextrate hydrate, xylitol and sorbitol, are found to be an oily or sticky mass, or a crystalline solid. A comparative data related to the formation and physical state of varenicline tartrate co-precipitates with different pharmaceutically acceptable excipients is furnished in the Example 5 as disclosed hereinafter.

The present inventors have now surprisingly and unexpectedly found amorphous co-precipitates of varenicline tartrate with a pharmaceutically acceptable excipient selected from the group consisting of maltodextrin, lactose monohydrate and 2-hydroxypropylβ-cyclodextrin, which have high purity, adequate stability and good dissolution properties.

The amorphous co-precipitates of varenicline tartrate disclosed herein are consistently reproducible, do not have the tendency to convert to crystalline forms, and are found to be stable. The amorphous co-precipitates of varenicline tartrate disclosed herein exhibit properties making them suitable for formulating varenicline tartrate.

More particularly, disclosed herein are amorphous co-precipitates of varenicline tartrate with improved physiochemical characteristics which help in the effective bioavailability of varenicline tartrate. Such pharmaceutical compositions may be administered easily to a mammalian patient in a dosage form, e.g., liquid, powder, elixir, injectable solution, with a high rate of bioavailability.

In yet another aspect, encompassed herein is a process for preparing the novel and stable amorphous co-precipitates of varenicline tartrate with pharmaceutically acceptable excipients, wherein the pharmaceutically acceptable excipient is selected from the group consisting of maltodextrin, lactose monohydrate and 2-hydroxypropyl-β-cyclodextrin.

In another aspect, provided herein are pharmaceutical compositions comprising the amorphous co-precipitates of varenicline tartrate and one or more pharmaceutically acceptable excipients.

In still further aspect, encompassed herein is a process for preparing pharmaceutical formulations comprising combining the amorphous co-precipitates of varenicline tartrate with one or more pharmaceutically acceptable excipients.

In another aspect, the amorphous co-precipitate of varenicline tartrate disclosed herein for use in the pharmaceutical compositions has a D₉₀ particle size of less than or equal to about 500 microns, specifically about 1 micron to about 300 microns, and most specifically about 5 microns to about 20 microns.

### BRIEF DESCRIPTION OF THE DRAWINGS

FIG. 1 is a characteristic powder X-ray diffraction (XRD) pattern of an amorphous co-precipitate of varenicline tartrate with maltodextrin (1:10).

FIG. 2 is a Scanning Electron Microscope (SEM) image of the morphological analysis of an amorphous co-precipitate of varenicline tartrate with maltodextrin (1:10).

FIG. 3 is a characteristic powder X-ray diffraction (XRD) pattern of an amorphous co-precipitate of varenicline tartrate with lactose monohydrate (1:10).

FIG. 4 is a Scanning Electron Microscope (SEM) image of the morphological analysis of an amorphous co-precipitate of varenicline tartrate with lactose monohydrate (1:10).

FIG. 5 is a characteristic powder X-ray diffraction (XRD) pattern of an amorphous co-precipitate of varenicline tartrate with 2-Hydroxypropyl-β-cyclodextrin (1:10).

FIG. 6 is a Scanning Electron Microscope (SEM) image of the morphological analysis of an amorphous co-precipitate of varenicline tartrate with 2-Hydroxypropyl-β-cyclodextrin (1:10).

### DETAILED DESCRIPTION

According to one aspect, there are provided stable amorphous co-precipitates comprising varenicline tartrate and a pharmaceutically acceptable excipient selected from the group consisting of maltodextrin, lactose monohydrate and 2-hydroxypropyl-β-cyclodextrin, having improved physiochemical characteristics that assist in the effective bioavailability of varenicline tartrate. A specific pharmaceutically acceptable excipient is maltodextrin.

The amorphous co-precipitates of varenicline tartrate with a pharmaceutically acceptable excipient obtained by the process disclosed herein are characterized by one or more of their powder X-ray diffraction (XRD) pattern, infrared absorption (IR) spectrum, and SEM images of the morphological analysis.

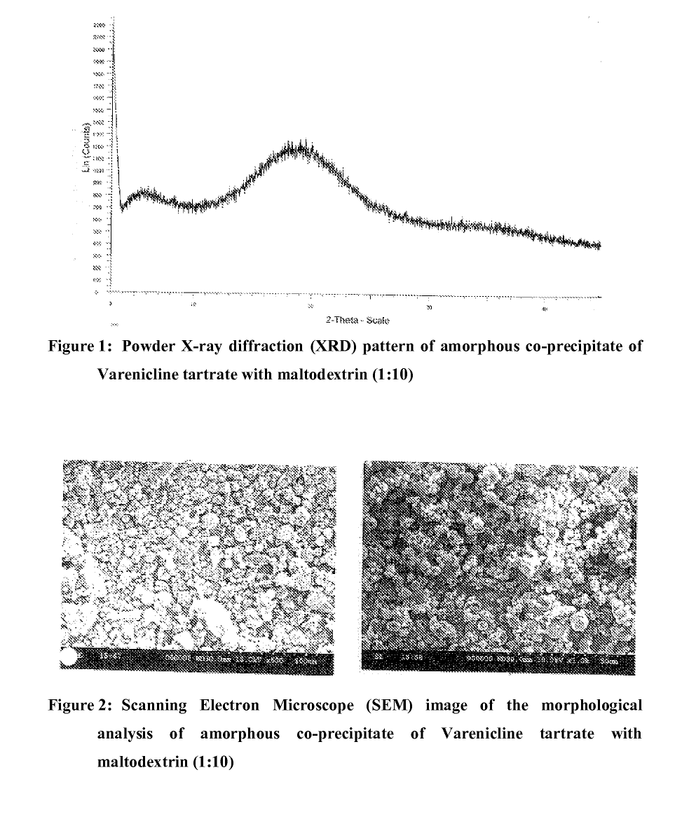

In one embodiment, provided herein is an amorphous co-precipitate of varenicline tartrate with maltodextrin, characterized by the following properties:
i) a powder X-ray diffraction pattern, showing a plain halo with no well-defined peaks, substantially in accordance with FIG. 1; and
ii) a Scanning Electron Microscope (SEM) image of the morphological analysis in accordance with FIG. 2.

In another embodiment, the amorphous co-precipitate of varenicline tartrate with maltodextrin disclosed herein remains in the same solid form and is stable when stored at a temperature of about 40±2° C. and at a relative humidity of about 75±5% for a period of at least 3 months.

In another embodiment, the amorphous co-precipitate of varenicline tartrate with maltodextrin disclosed herein remains in the same solid form and is stable when stored at a temperature of about 30±2° C. and at a relative humidity of about 65±5% for a period of at least 3 months.

In another embodiment, the amorphous co-precipitate of varenicline tartrate with maltodextrin disclosed herein remains in the same solid form and is stable when stored at a temperature of about 25±2° C. and at a relative humidity of about 60±5% for a period of at least 3 months.

In another embodiment, the amorphous co-precipitate of varenicline tartrate with maltodextrin disclosed herein remains in the same solid form and is stable when stored at a temperature of about 2° C. to 8° C. for a period of at least 3 months.

The term “remains stable”, as defined herein, refers to lack of formation of impurities, while being stored as described hereinbefore.

Moreover, the amorphous co-precipitate of varenicline tartrate with maltodextrin has a tapped density of at least about 0.5 g/ml, and specifically about 0.60 g/ml to about 0.7 g/ml, and which is particularly suitable for bulk preparation and handling. So, the amorphous co-precipitate of varenicline tartrate with maltodextrin disclosed herein is suitable for formulating varenicline tartrate.

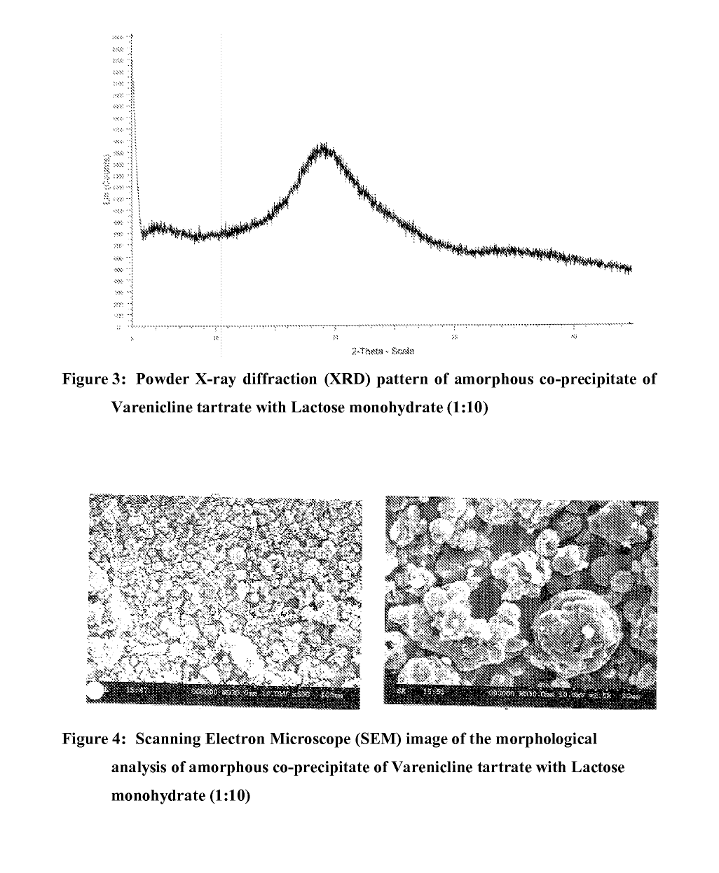

In another embodiment, provided herein is an amorphous co-precipitate of varenicline tartrate with lactose monohydrate, characterized by the following properties:
i) a powder X-ray diffraction pattern, showing a plain halo with no well-defined peaks, substantially in accordance with FIG. 3; and
ii) a Scanning Electron Microscope (SEM) image of the morphological analysis in accordance with FIG. 4.

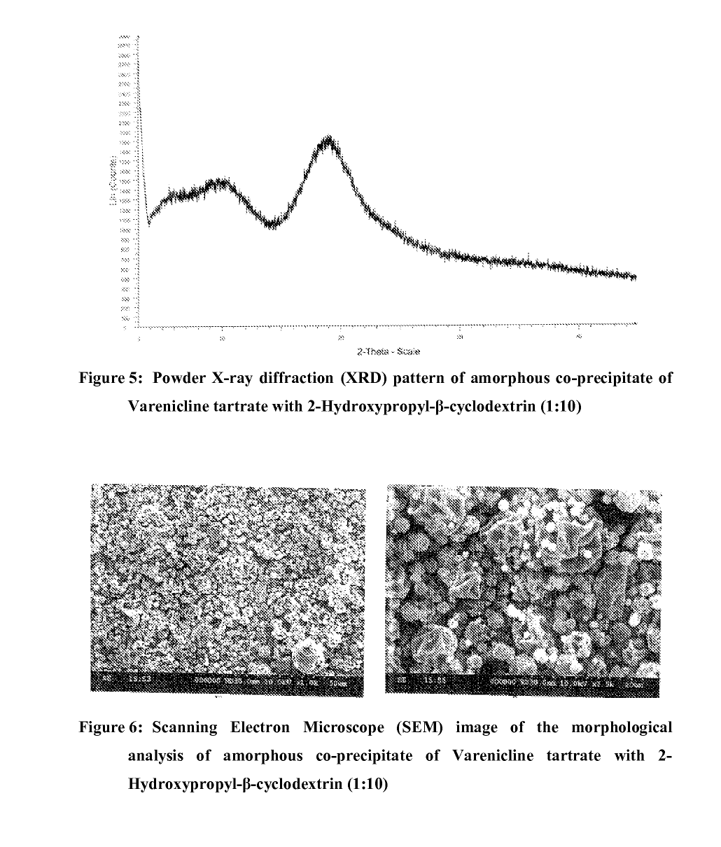

In another embodiment, provided herein is an amorphous co-precipitate of varenicline tartrate with 2-hydroxypropyl-β-cyclodextrin, characterized by the following properties:
i) a powder X-ray diffraction pattern, showing a plain halo with no well-defined peaks, substantially in accordance with FIG. 5; and
ii) a Scanning Electron Microscope (SEM) image of the morphological analysis in accordance with FIG. 6.

According to another aspect, there is provided a process for the preparation of an amorphous coprecipitate of varenicline tartrate and a pharmaceutically acceptable excipient selected from the group consisting of maltodextrin, lactose monohydrate and 2-hydroxypropyl-β-cyclodextrin, comprising:
a) providing a solution of varenicline tartrate and a pharmaceutically acceptable excipient in a solvent, wherein the pharmaceutically acceptable excipient is selected from the group consisting of maltodextrin, lactose monohydrate and 2-hydroxypropyl-β-cyclodextrin, and wherein the solvent is water, an organic solvent or a solvent medium comprising water and an organic solvent;
b) optionally, filtering the solution to remove insoluble matter; and
c) substantially removing the solvent from the solution to afford amorphous coprecipitate of varenicline tartrate with the pharmaceutically acceptable excipient.

The process can produce amorphous co-precipitates of varenicline tartrate with a pharmaceutically acceptable excipient in substantially pure form.

The term “substantially pure amorphous co-precipitate of varenicline tartrate with a pharmaceutically acceptable excipient” refers to the amorphous co-precipitate of varenicline tartrate having purity greater than about 99%, specifically greater than about 99.5%, more specifically greater than about 99.8% and still more specifically greater than about 99.9% (measured by HPLC).

In one embodiment, the preferred pharmaceutically acceptable excipient used in step-(a) is maltodextrin.

Exemplary organic solvents used in step-(a) include, but are not limited to, an alcohol, a ketone, a nitrile, and mixtures thereof. The term solvent also includes mixtures of solvents.

In one embodiment, the organic solvent is selected from the group consisting of methanol, ethanol, n-propanol, isopropyl alcohol, isobutanol, n-butanol, tert-butanol, amyl alcohol, isoamyl alcohol, acetone, methyl ethyl ketone, methyl isobutyl ketone, methyl tert-butyl ketone, acetonitrile, and mixtures thereof. Specifically, the organic solvent is selected from the group consisting of methanol, ethanol, isopropyl alcohol, acetone, and mixtures thereof.

Step-(a) of providing a solution includes dissolving a crystalline or amorphous form of varenicline tartrate in the solvent, or such a solution may be obtained directly from a reaction in which varenicline tartrate is formed; and combining the solution with a pharmaceutically acceptable excipient. In one embodiment, the pharmaceutical excipient can be dissolved in a solution containing varenicline tartrate, or, varenicline tartrate can be dissolved in a solution containing a pharmaceutical excipient.

Alternatively, a solution containing varenicline tartrate can be combined with a solution containing a pharmaceutically acceptable excipient, and the solvents used for preparing the different solutions need not be the same as long as the solvents have mutual solubility and form a single phase. In any event, varenicline tartrate should be completely soluble in the solvents used and should provide a clear solution. The presence of undissolved crystals could lead to the formation of a material that is not completely amorphous.

In one embodiment, the dissolution is carried out at a temperature of about 0° C. to about 140° C., specifically at about 20° C. to about 100° C., and more specifically at about 25° C. to about 80° C.

In another embodiment, the solution in step-(a) is prepared by admixing varenicline base, L-tartaric acid and the solvent to obtain a mixture; stirring the mixture to obtain a solution of varenicline tartrate; and combining the solution with a pharmaceutically acceptable excipient. In yet another embodiment, the mixture is stirred at a temperature of below about 0° C. to about 140° C. for at least 15 minutes, specifically at about 20° C. to about 100° C. for about 20 minutes to about 10 hours, and still more specifically at about 25° C. to about 80° C. for about 30 minutes to about 2 hours.

In one embodiment, the L-tartaric acid is used directly, in the form of an aqueous solution of L-tartaric acid or in the form of L-tartaric acid dissolved in an organic solvent. The organic solvent used for dissolving L-tartaric acid is selected from the group as described above.

In another embodiment, the solution obtained in step-(a) is optionally subjected to carbon treatment or silica gel treatment. The carbon treatment or silica gel treatment is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing varenicline tartrate and a pharmaceutically acceptable excipient by removing charcoal or silica gel. Preferably, a finely powdered carbon is an active carbon. In one embodiment, a specific mesh size of silica gel is 40-500 mesh, and more specifically 60-120 mesh.

The solution obtained in step-(a) is optionally stirred at a temperature of about 20° C. to the reflux temperature of the solvent used for at least 20 minutes, and specifically at a temperature of about 40° C. to the reflux temperature of the solvent used for about 30 minutes to about 4 hours.

As used herein, “reflux temperature” means the temperature at which the solvent or solvent system refluxes or boils at atmospheric pressure.

The term “substantially removing” the solvent refers to at least 80%, specifically greater than about 85%, more specifically greater than about 90%, still more specifically greater than about 99%, and most specifically essentially complete (100%), removal of the solvent from the solvent solution.

Removal of solvent in step-(c) is accomplished, for example, by substantially complete evaporation of the solvent, concentrating the solution, or distillation of solvent, under inert atmosphere to obtain amorphous co-precipitate comprising varenicline tartrate and a pharmaceutically acceptable excipient.

In one embodiment, the solvent is removed by evaporation. Evaporation can be achieved at sub-zero temperatures by lyophilisation or freeze-drying techniques. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

The distillation process can be performed at atmospheric pressure or reduced pressure. Specifically, the solvent is removed at a pressure of about 760 mm Hg or less, more specifically at about 400 mm Hg or less, still more specifically at about 80 mm Hg or less, and most specifically from about 30 to about 80 mm Hg.

Solvents can also be removed by spray-drying, in which a solution of varenicline tartrate and a pharmaceutically acceptable excipient is sprayed into the spray drier at the flow rate ranging from 10 to 300 ml/hr, specifically 40 to 200 ml/hr. The air inlet temperature to the spray drier used may range from about 30° C. to about 150° C., specifically from about 65° C. to about 110° C. and the outlet air temperature used may range from about 30° C. to about 90° C.

Another suitable method is vertical agitated thin-film drying (or evaporation). Agitated thin film evaporation technology involves separating the volatile component using indirect heat transfer coupled with mechanical agitation of the flowing film under controlled conditions. In vertical agitated thin-film drying (or evaporation) (ATFD-V), the starting solution is fed from the top into a cylindrical space between a centered rotary agitator and an outside heating jacket. The rotor rotation agitates the downside-flowing solution while the heating jacket heats it.

In one embodiment, the coprecipitate of varenicline tartrate with the pharmaceutically acceptable excipient obtained in step-(c) is recovered by methods such as filtration, filtration under vacuum, decantation, centrifugation, or a combination thereof. In another embodiment, the coprecipitate of varenicline tartrate is recovered by filtration employing a filtration media of, for example, a silica gel or celite.

The pure coprecipitate of varenicline tartrate with the pharmaceutically acceptable excipient obtained by above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 25° C. to about 80° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing conditions should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The dried product obtained by the process disclosed herein above can optionally be milled to get desired particle sizes. Milling or micronization can be performed prior to drying, or after the completion of drying of the product. The milling operation reduces the size of particles and increases surface area of particles. Drying is more efficient when the particle size of the material is smaller and the surface area is higher, hence milling will frequently be performed prior to the drying operation.

Milling can be done suitably using jet milling equipment like an air jet mill, or using other conventional milling equipment.

The resulting amorphous powder compositions disclosed herein have improved solubility properties and hence also have improved bioavailability.

The amorphous co-precipitates of varenicline tartrate with the pharmaceutically acceptable excipients obtained by the process disclosed herein are a random distribution of the varenicline tartrate and the pharmaceutically acceptable excipient in a particle matrix. Without being held to any particular theory, the co-precipitates have the characteristics of solid dispersions at a molecular level, being in the nature of solid solutions. The solid solutions, or molecular dispersions, provide homogeneous particles in which no discrete areas of only amorphous varenicline tartrate and/or only pharmaceutically acceptable excipient can be observed.

Further encompassed herein is the use of the amorphous coprecipitate of varenicline tartrate and a pharmaceutically acceptable excipient selected from the group consisting of maltodextrin, lactose monohydrate and 2-hydroxypropyl-β-cyclodextrin for the manufacture of a pharmaceutical composition together with a pharmaceutically acceptable carrier.

A specific pharmaceutical composition of the amorphous coprecipitate of varenicline tartrate and a pharmaceutically acceptable excipient is selected from a solid dosage form and an oral suspension.

In one embodiment, the amorphous coprecipitate of varenicline tartrate and a pharmaceutically acceptable excipient has a D₉₀ particle size of less than or equal to about 500 microns, specifically about 1 micron to about 300 microns, and most specifically about 5 microns to about 20 microns, wherein the pharmaceutically acceptable excipient is selected from the group consisting of maltodextrin, lactose monohydrate and 2-hydroxypropyl-β-cyclodextrin.

In another embodiment, the substantially pure amorphous coprecipitate of varenicline tartrate with a pharmaceutically acceptable excipient disclosed herein for use in the pharmaceutical compositions has a D₉₀ particle size of less than or equal to about 500 microns, specifically about 1 micron to about 300 microns, and most specifically about 5 microns to about 20 microns.

In another embodiment, the particle sizes of the amorphous coprecipitate of varenicline tartrate and a pharmaceutically acceptable excipient can be achieved by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for treating a patient suffering from diseases caused by neurogical and psychological disorders, inflammatory bowel disease, irritable bowel syndrome, spastic dystonia, chronic pain, acute pain, vasoconstriction, anxiety, panic disorder, depression, cognitive dysfunction, drug/toxin-induced cognitive impairment, nicotine dependency and addiction; comprising administering a therapeutically effective amount of the amorphous coprecipitate of varenicline tartrate and a pharmaceutically acceptable excipient selected from the group consisting of maltodextrin, lactose monohydrate and 2-hydroxypropyl-β-cyclodextrin, or a pharmaceutical composition that comprises a therapeutically effective amount of the amorphous coprecipitate of varenicline tartrate and a pharmaceutically acceptable excipient, along with pharmaceutically acceptable excipients.

According to another aspect, there are provided pharmaceutical compositions comprising amorphous coprecipitate of varenicline tartrate and a pharmaceutically acceptable excipient disclosed herein and one or more pharmaceutically acceptable excipients.

According to another aspect, there is provided a process for preparing a pharmaceutical formulation comprising combining the amorphous coprecipitate of varenicline tartrate with a pharmaceutically acceptable excipient disclosed herein, with one or more pharmaceutically acceptable excipients.

Yet in another embodiment, pharmaceutical compositions comprise at least a therapeutically effective amount of amorphous coprecipitate of varenicline tartrate with a pharmaceutically acceptable excipient selected from the group consisting of maltodextrin, lactose monohydrate and 2-hydroxypropyl-β-cyclodextrin. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The amorphous coprecipitate of varenicline tartrate with a pharmaceutically acceptable excipient selected from the group consisting of maltodextrin, lactose monohydrate and 2-hydroxypropyl-β-cyclodextrin may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described herein.

In one embodiment, capsule dosage forms contain amorphous coprecipitate of varenicline tartrate with a pharmaceutically acceptable excipient selected from the group consisting of maltodextrin, lactose monohydrate and 2-hydroxypropyl-β-cyclodextrin within a capsule which may be coated with gelatin. Tablets and powders may also be coated with an enteric coating. Suitable enteric coating agents include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### INSTRUMENTAL DETAILS

### X-Ray Powder Diffraction (P-XRD)

The X-Ray powder diffraction was measured by an X-ray powder Diffractometer equipped with CuKα-radiations (40 kV, 40 mA) in wide-angle X-ray Diffractometer of BRUKER axs, D8 ADVANCE. The sample was analyzed using the following instrument parameters: measuring range=3-45° 2-theta; step width=0.01579°; and measuring time per step=0.11 seconds.

The following examples are given for the purpose of illustrating the present disclosure and should not be considered as limitation on the scope or spirit of the disclosure.

### EXAMPLES

### Example 1

### Preparation of Amorphous Coprecipitate of Varenicline Tartrate With Maltodextrin

Varenicline free base (0.5 g) was dissolved in methanol (5 ml) at 25-30° C., followed by the addition of a solution of tartaric acid (0.355 g, dissolved in 5 ml water) at 25-30° C. The clear solution was stirred for 30 minutes at 25-30° C. This was followed by the addition of a solution of maltodextrin (8.55 g) in water (30 ml) at 25-30° C. The clear solution was stirred for 30 minutes, followed by filtration through a hyflow bed and washing the bed with water (10 ml). The clear filtrate was subjected to spray drying using a spray dryer (Jay instrument, JISL, LSD-48 mini spray dryer) under the conditions of inlet temperature at 125° C., outlet temperature at 67-75° C., aspirator-70 and feed pump-30%. The resulting amorphous coprecipitate of varenicline tartrate with maltodextrin was recovered under nitrogen atmosphere (Purity by HPLC: 99.85%).

### Example 2

### Preparation of Amorphous Coprecipitate of Varenicline Tartrate With Maltodextrin (1:10)

Varenicline free base (2 g) was dissolved in methanol (20 ml) at 25-30° C., and tartaric acid solution (1.4 g, dissolved in 20 ml water) was added at 25-30° C. The resulting clear solution was stirred for 30 min at 25-30° C. This was followed by the addition of a solution of maltodextrin (34.1 g) dissolved in water (140 ml) at 25-30° C. The clear solution was stirred for 30 minutes, filtered through a hyflow bed and washed with water (20 ml). The clear filtrate was subjected to spray drying using a spray dryer [Jay instrument, JISL, LSD-48 Mini spray dryer] under the following conditions to give the amorphous coprecipitate of varenicline tartrate with maltodextrin [Purity by HPLC: 99.85%; Particle size distribution: d(0.9)=11 to 13 microns; Tapped Density: 0.5 to 0.7 g/ml].

Conditions of Spray Drying: Feed pump=30; Inlet temp=125° C.; Outlet=67-75° C. and Aspirator=70.

Stability: The product obtained was found to be stable for at least 3 months at a temperature of about 40±2° C. under a relative humidity of about 75±5%, at a temperature of about 30±2° C. under a relative humidity of about 65±5%, at a temperature of about 25±2° C. under a relative humidity of about 60±5%, and at a temperature of about 2° C. to 8° C., when stored in a three bag packing system wherein the inner bag is an antistatic translucent polyethylene bag kept with heat seal under vacuum and nitrogen atmosphere, the middle bag is an antistatic translucent polyethylene bag containing one silicagel pouch and heat seal, and the outer bag is a triple laminated aluminum liner bag kept with heat seal under vacuum and nitrogen atmosphere.

### Example 3

### Preparation of Amorphous Coprecipitate of Varenicline Tartrate With Lactose Monohydrate (1:10)

Varenicline free base (0.5 g) was dissolved in methanol (5 ml) at 25-30° C., and tartaric acid solution (0.355 g, dissolved in 5 ml water) was added at 25-30° C. The clear solution was stirred for 30 minutes at 25-30° C. This was followed by the addition of lactose monohydrate (8.55 g) dissolved in water (85 ml) at 25-30° C. The resulting clear solution was stirred for 30 minutes, and filtered through a hyflow bed and washed with (1:1) mixture of methanol: water (10 ml). The clear filtrate was subjected to spray drying using a spray dryer [Jay instrument, JISL, LSD -48 Mini spray dryer] under the following conditions to give the amorphous coprecipitate of varenicline tartrate with lactose monohydrate (Purity by HPLC: 99.85%).

Conditions of Spray Drying: Feed pump=30; Inlet temp=125° C.; Outlet=67-75° C. and Aspirator=70.

### Example 4

### Preparation of Amorphous Coprecipitate of Varenicline Tartrate With 2-hydroxypropyl-β-cyclodextrin (1:10)

Varenicline free base (0.5 g) was dissolved in methanol (5 ml) at 25-30° C. and tartaric acid solution (0.355 g, dissolved in 5 ml water) was added at 25-30° C. The clear solution was stirred for 30 minutes at 25-30° C. This was followed by the addition of 2-hydroxypropyl-β-cyclodextrin (8.55 g) dissolved in a (1:1) mixture of methanol: water (40 ml) at 25-30° C. The clear solution was stirred for 30 minutes, and filtered through a hyflow bed and washed with 1:1 methanol:water (10 ml). The clear filtrate was subjected to spray drying using a spray dryer [Jay instrument, JISL, LSD-48 mini spray dryer using the following conditions to give the amorphous coprecipitate of varenicline tartrate with 2-hydroxypropyl-β-cyclodextrin (Purity by HPLC: 99.7%).

Conditions of Spray Drying: Feed pump=30; Inlet temp=125° C.; Outlet=67-75° C. and Aspirator=70.

### Example 5

### Co-Precipitate Samples Prepared Using Different Excipients In Different Ratios

The comparative data related to the formation and physical state of varenicline tartrate co-precipitates with different pharmaceutically acceptable excipients is furnished in the below table.

| | Co-precipitate Sample | | Physical State | |
|---|---|---|---|---|
| S. No. | Name | Batch size and Ratio | (by P-XRD) | Yield (g) |
| 1 | Varenicline tartrate with | 0.85 g & 8.5 g (1:10) | Amorphous solid | 2 g |
| | Povidone K30 | | | |
| 2 | Varenicline tartrate with | 0.85 g & 17 g (1:20) | Amorphous solid | 3 g |
| | Povidone K30 | | | |
| 3 | Varenicline tartrate with | 5.13 g & 5.13 g (1:1) | Amorphous solid | 3.5 g |
| | Povidone K30 | | | |
| 4 | Varenicline tartrate with | 0.85 g & 8.5 g (1:10) | Amorphous solid | 2.8 g |
| | Lactose monohydrate | | | |
| 5 | Varenicline tartrate with | 0.85 g & 8.5 g (1:10) | Crystalline Solid | 2.4 g |
| | Mannitol | | | |
| 6 | Varenicline tartrate with | 0.85 g & 8.5 g (1:10) | Amorphous solid | 3.2 g |
| | Maltodextrin | | | |
| 7 | Varenicline tartrate with | 0.85 g & 8.5 g (1:10) | Oily and sticky | — |
| | D-Maltitol | | mass | |
| 8 | Varenicline tartrate with | 0.85 g & 8.5 g (1:10) | Amorphous solid | 4.8 g |
| | 2-Hydroxypropyl-β- | | | |
| | cyclodextrin | | | |
| 9 | Varenicline tartrate with | 0.85 g & 8.5 g (1:10) | Oily and sticky | — |
| | Dextrate hydrate | | mass | |
| 10 | Varenicline tartrate with | 0.85 g & 8.5 g (1:10) | Oily and sticky | — |
| | Xylitol | | mass | |
| 11 | Varenicline tartrate with | 0.85 g & 8.5 g (1:10) | Oily and sticky | — |
| | Sorbitol | | mass | |

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable, and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such materials known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP).

The term “micronization” used herein means a process or method by which the size of a population of particles is reduced.

As used herein, the term “micron” or “μm” both are equivalent and refer to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of less than 300 microns means that 90 volume-percent of the particles in a composition have a diameter less than 300 microns.

The term “coprecipitate or co-precipitate” as used herein refers to compositions comprising amorphous varenicline tartrate together with at least one pharmaceutically acceptable excipient, being prepared by removing solvent from a solution containing both of them.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An amorphous coprecipitate comprising varenicline tartrate and a pharmaceutically acceptable excipient selected from the group consisting of maltodextrin, lactose monohydrate and 2-hydroxypropyl-β-cyclodextrin; having the following characteristics, wherein:
a) the amorphous coprecipitate of varenicline tartrate with maltodextrin is characterized by a powder X-ray diffraction pattern showing a plain halo with no well-defined peaks substantially in accordance with FIG. 1, and a scanning electron microscope (SEM) image of the morphological analysis in accordance with FIG. 2;
b) the amorphous co-precipitate of varenicline tartrate with lactose monohydrate is characterized by a powder X-ray diffraction pattern showing a plain halo with no well-defined peaks substantially in accordance with FIG. 3, and a Scanning Electron Microscope (SEM) image of the morphological analysis in accordance with FIG. 4; and
c) the amorphous co-precipitate of varenicline tartrate with 2-hydroxypropyl-β-cyclodextrin is characterized by a powder X-ray diffraction pattern showing a plain halo with no well-defined peaks substantially in accordance with FIG. 5; and a Scanning Electron Microscope (SEM) image of the morphological analysis in accordance with FIG. 6.

2. A process for the preparation of the amorphous coprecipitate of claim 1, comprising:
a) providing a solution of varenicline tartrate and a pharmaceutically acceptable excipient in a solvent, wherein the pharmaceutically acceptable excipient is selected from the group consisting of maltodextrin, lactose monohydrate and 2-hydroxypropyl-β-cyclodextrin; and wherein the solvent is water, an organic solvent or a solvent medium comprising water and an organic solvent, wherein the organic solvent is selected from the group consisting of an alcohol, a ketone, a nitrile, and mixtures thereof;
b) optionally, filtering the solution to remove insoluble matter; and
c) substantially removing the solvent from the solution to afford the amorphous coprecipitate of varenicline tartrate with a pharmaceutically acceptable excipient.

3. The process of claim 2, wherein the organic solvent is selected from the group consisting of methanol, ethanol, n-propanol, isopropyl alcohol, isobutanol, n-butanol, tert-butanol, amyl alcohol, isoamyl alcohol, acetone, methyl ethyl ketone, methyl isobutyl ketone, methyl tert-butyl ketone, acetonitrile, and mixtures thereof; and wherein the pharmaceutically acceptable excipient is maltodextrin.

4. The process of claim 3, wherein the organic solvent is methanol.

5. The process of claim 2, wherein the solution in step-(a) is provided by i) dissolving varenicline tartrate in the solvent, followed by combining the solution with the pharmaceutical excipient; or ii) dissolving the pharmaceutical excipient in the solvent, followed by combining the solution with varenicline tartrate; or iii) combining the solution containing varenicline tartrate with a solution containing the pharmaceutically acceptable excipient; or iv) admixing varenicline base, L-tartaric acid and the solvent to obtain a mixture, stirring the mixture to obtain a solution of varenicline tartrate, and combining the solution with a pharmaceutically acceptable excipient.

6. The process of claim 5, wherein the dissolution is carried out at a temperature of about 0° C. to about 140° C.

7. The process of claim 6, wherein the dissolution is carried out at a temperature of about 25° C. to about 80° C.

8. The process of claim 2, wherein the solution obtained in step-(a) is optionally subjected to carbon treatment or silica gel treatment; wherein the removal of the solvent in step-(c) is accomplished by distillation or complete evaporation of the solvent, spray drying, vacuum drying, lyophilization or freeze drying, agitated thin-film drying, or a combination thereof; and wherein the substantially pure coprecipitate of varenicline tartrate obtained in step-(c) is further dried under vacuum or at atmospheric pressure, at a temperature of about 35° C. to about 80° C.

9. A pharmaceutical composition comprising the amorphous coprecipitate of varenicline tartrate of claim 1, and one or more pharmaceutically acceptable excipients.

10. The pharmaceutical composition of claim 9, wherein the pharmaceutical composition is a solid dosage form, an oral suspension, a liquid, a powder, an elixir, an aerosol, syrups or an injectable solution.

11. The pharmaceutical composition of claim 9, wherein the amorphous coprecipitate of varenicline tartrate has a D₉₀ particle size of less than or equal to about 500 microns.

12. The pharmaceutical composition of claim 11, wherein the D₉₀ particle size is about 1 micron to about 300 microns, or about 5 microns to about 20 microns.

13. A method for treating a patient suffering from diseases caused by neurogical and psychological disorders, inflammatory bowel disease, irritable bowel syndrome, spastic dystonia, chronic pain, acute pain, vasoconstriction, anxiety, panic disorder, depression, cognitive dysfunction, drug/toxin-induced cognitive impairment, nicotine dependency and addiction; comprising administering a therapeutically effective amount of the amorphous coprecipitate of varenicline tartrate of claim 1, or a pharmaceutical composition that comprises a therapeutically effective amount of amorphous coprecipitate of varenicline tartrate of claim 9.
